(19) United States
(12) Patent Application Publication
(10) Pub. No.: US 2026/0162770 A1
(43) Pub. Date: Jun. 11, 2026

(54) METHOD AND DEVICE FOR ANALYZING SINGLE CELL RNA SEQUENCING DATA

(71) Applicant: SEOUL NATIONAL UNIVERSITY R&DB FOUNDATION, Seoul (KR)

(72) Inventors: Buhm HAN, Seoul (KR); Hanbin LEE, Seoul (KR)

(73) Assignee: SEOUL NATIONAL UNIVERSITY R&DB FOUNDATION, Seoul (KR)

(21) Appl. No.: 18/721,180

(22) PCT Filed: Dec. 16, 2022
(86) PCT No.: PCT/KR2022/020542

§ 371 Date: Oct. 22, 2024

(30) Foreign Application Priority Data
Dec. 17, 2021 (KR) 10-2021-0182148
May 9, 2022 (KR) 10-2022-0056597
Oct. 18, 2022 (KR) 10-2022-0134189

(51) Int. Cl. G16B 25/10; G16B 5/20; G16B 30/10

(52) U.S. Cl. CPC G16B 25/10; G16B 5/20; G16B 30/10

(57) ABSTRACT

A method and a device for analyzing single cell RNA sequencing data and a computer program are disclosed. A data analysis method according to one embodiment may comprise the steps of: acquiring, in correspondence to each of a plurality of batches, property information quantitatively measured in a single cell; calculating, on the basis of the sum of property information about cells corresponding to each of the plurality of batches, a conditional expectation and a conditional dispersion of property information modeled on a poisson distribution; calculating, on the basis of the conditional expectation, the conditional dispersion, and the property information, a covariance matrix of a residual matrix corresponding to the property information; and performing eigen-decomposition on the covariance matrix so as to calculate a PC value for PCA of the residual matrix.

### TECHNICAL FIELD

The following embodiments relate to a data analysis method and device for single-cell RNA sequencing and, more particularly, to an analysis method and device for dimensionality reduction of single-cell RNA sequencing data.

### RELATED ART

Single-cell RNA sequencing (scRNA-seq), or single-cell transcriptomics, refers to a technique that isolates a single cell and amplifies and sequences RNA from an extremely small amount of material to analyze the genomic characteristics of the cell. As a result of single-cell RNA sequencing, single-cell RNA sequencing data or single-cell transcriptomics data, which indicates a transcript expression level corresponding to each gene in the cell, may be acquired. For example, an analysis of single-cell transcriptomics data may involve a principal component analysis (PCA) to generate low-dimensional embeddings. A result of the PCA on single-cell transcriptomics data may be used for two-dimensional (2D) transformation (e.g., tSNE or UMAP)-based cell distribution visualization, unsupervised clustering algorithm-based cell clustering, or the like.

### SUMMARY

### Technical Solutions

According to an embodiment of the present disclosure, there is provided a data analysis method including: acquiring characteristic information quantitatively measured from a single cell, corresponding to each of a plurality of batches; calculating a conditional expectation and a conditional variance of the characteristic information modeled to a Poisson distribution, based on a sum of the characteristic information of cells corresponding to each of the plurality of batches; calculating a covariance matrix of a residual matrix corresponding to the characteristic information, based on the conditional expectation, the conditional variance, and the characteristic information; and calculating a PC value for a principal component analysis (PCA) of the residual matrix through eigen-decomposition of the covariance matrix.

The calculating of the conditional expectation and the conditional variance may include: modeling the characteristic information to a multinomial distribution conditional on the sum of the characteristic information of the cells corresponding to each of the plurality of batches; and calculating the conditional expectation and the conditional variance based on the multinomial distribution of the characteristic information.

The calculating of the covariance matrix of the residual matrix may include: calculating a sum of AA^{T}, 2AB^{T}, and BB^{T}, wherein A denotes Y_{bij}/σ_{bij}; B denotes μ_{bij}/σ_{bij}; Y_{bij} denotes characteristic information corresponding to batch b, cell i, and gene j; μ_{bij} denotes a conditional expectation of Y_{bij}; and σ_{bij} denotes a conditional variance of Y_{bij}.

The residual matrix may be equivalent to a matrix obtained by subtracting B from A.

The covariance matrix of the residual matrix may be equivalent to a product of the residual matrix and a transposed matrix of the residual matrix.

AB^{T} and BB^{T} may be calculated as expressed in the following equation.

$${n}_{bi} = {\sum }_{j} {Y}_{bij} , {n}_{bj} = {\sum }_{i ∈ {Φ}_{b}} {Y}_{bij} , {n}_{\text{?}} = {\sum }_{i ∈ {Φ}_{b} ,} {Y}_{bij} \qquad [ Equation ]$$

$${p}_{\text{?}} = {n}_{\text{?}} / {n}_{\text{?}} .$$

$$\begin{matrix}{AB}_{{jj}^{′}}^{T} = {\sum }_{b = 1}^{{n}_{B}} {\sum }_{k ∈ {Φ}_{b}} {A}_{bkj} {B}_{{bkj}^{′}} \\ = {\sum }_{b = 1}^{{n}_{B}} {\sum }_{k ∈ {Φ}_{b}} {A}_{bkj} \sqrt{{n}_{{bj}^{′}} \frac{{p}_{bk}}{1 - {p}_{bk}}} \\ = {\sum }_{b = 1}^{{n}_{B}} \sqrt{{n}_{{bj}^{′}}} {\sum }_{k ∈ {Φ}_{b}} {A}_{bkj} \sqrt{\frac{{p}_{bk}}{1 - {p}_{bk}}}\end{matrix}$$

$${BB}_{{jj}^{′}}^{T} = {\sum }_{b = 1}^{{n}_{B}} \sqrt{{n}_{bj} {n}_{{bj}^{′}}} {\sum }_{k ∈ {Φ}_{b}} \frac{{p}_{bk}}{1 - {p}_{bk}}$$

$$\text{?} \text{indicates text missing or illegible when filed}$$

The calculating of the covariance matrix of the residual matrix may include: calculating a variance of a residual corresponding to each gene in the characteristic information, based on the conditional expectation, the conditional variance, and the characteristic information; determining a subset of the characteristic information including at least a portion of genes included in the characteristic information, based on the variance of the residual corresponding to each gene; and calculating a covariance matrix of a residual matrix corresponding to the determined subset.

The determining of the subset of the characteristic information may include: selecting a predetermined number of genes in an order of increasing variance of the residual; and determining the subset of the characteristic information including the selected genes.

The calculating of the PC value of the residual matrix may include: acquiring an eigenvector corresponding to an eigen-space of the residual matrix by performing eigen-decomposition on the covariance matrix of the residual matrix; and calculating the PC value of the residual matrix, which is a projection of the residual matrix onto the eigen-space, based on the eigenvector.

The characteristic information may include at least one of: RNA sequencing data including a transcript expression level corresponding to each gene in each cell; data on a quantity of transcription factors bound to DNA for each cell; and open DNA sequencing data for each cell.

The characteristic information may be a sparse matrix.

Each of the plurality of batches may correspond to a donor of cells included in the characteristic information.

According to an embodiment of the present disclosure, there is provided a device including: at least one processor configured to acquire characteristic information quantitatively measured from a single cell, corresponding to each of a plurality of batches; calculate a conditional expectation and a conditional variance of the characteristic information modeled to a Poisson distribution, based on a sum of the characteristic information of cells corresponding to each of the plurality of batches; calculate a covariance matrix of a residual matrix corresponding to the characteristic information, based on the conditional expectation, the conditional variance, and the characteristic information; and calculate a PC value for a PCA of the residual matrix through eigen-decomposition of the covariance matrix.

For calculating the conditional expectation and the conditional variance, the processor may be configured to model the characteristic information to a multinomial distribution conditional on the sum of the characteristic information of the cells corresponding to each of the plurality of batches; and calculate the conditional expectation and the conditional variance based on the multinomial distribution of the characteristic information.

For calculating the covariance matrix of the residual matrix, the processor may be configured to calculate a sum of AA^{T}, 2AB^{T}, and BB^{T}, wherein A denotes Y_{bij}/σ_{bij}; B denotes μ_{bij}/σ_{bij}; Y_{bij} denotes characteristic information corresponding to batch b, cell i, and gene j; μ_{bij} denotes a conditional expectation of Y_{bij}; and σ_{bij} denotes a conditional variance of Y_{bij}.

The residual matrix may be equivalent to a matrix obtained by subtracting B from A.

AB^{T} and BB^{T} may be calculated as expressed in the following equation.

$${n}_{bi} = {\sum }_{j} {Y}_{bij} , {n}_{bj} = {\sum }_{i ∈ {Φ}_{b}} {Y}_{bij} , {n}_{\text{?}} = {\sum }_{i ∈ {Φ}_{b} ,} {Y}_{bij} \qquad [ Equation ]$$

$${p}_{\text{?}} = {n}_{\text{?}} / {n}_{\text{?}} .$$

$$\begin{matrix}{AB}_{{jj}^{′}}^{T} = {\sum }_{b = 1}^{{n}_{B}} {\sum }_{k ∈ {Φ}_{b}} {A}_{bkj} {B}_{{bkj}^{′}} \\ = {\sum }_{b = 1}^{{n}_{B}} {\sum }_{k ∈ {Φ}_{b}} {A}_{bkj} \sqrt{{n}_{{bj}^{′}} \frac{{p}_{bk}}{1 - {p}_{bk}}} \\ = {\sum }_{b = 1}^{{n}_{B}} \sqrt{{n}_{{bj}^{′}}} {\sum }_{k ∈ {Φ}_{b}} {A}_{bkj} \sqrt{\frac{{p}_{bk}}{1 - {p}_{bk}}}\end{matrix}$$

$${BB}_{{jj}^{′}}^{T} = {\sum }_{b = 1}^{{n}_{B}} \sqrt{{n}_{bj} {n}_{{bj}^{′}}} {\sum }_{k ∈ {Φ}_{b}} \frac{{p}_{bk}}{1 - {p}_{bk}}$$

$$\text{?} \text{indicates text missing or illegible when filed}$$

For calculating the covariance matrix of the residual matrix, the processor may be configured to calculate a variance of a residual corresponding to each gene in the characteristic information, based on the conditional expectation, the conditional variance, and the characteristic information; determine a subset of the characteristic information including at least a portion of genes included in the characteristic information, based on the variance of the residual corresponding to each gene; and calculate a covariance matrix of a residual matrix corresponding to the determined subset.

For determining the subset of the characteristic information, the processor may be configured to select a predetermined number of genes in an order of increasing variance of the residual; and determine the subset of the characteristic information including the selected genes.

### BRIEF DESCRIPTION OF THE DRAWINGS

FIG. 1 is a diagram illustrating an example framework of a data analytics model according to an embodiment.

FIG. 2 is a flowchart illustrating the flow of steps of a data analysis method according to an embodiment.

FIG. 3 is a diagram illustrating a data analysis method including a gene selection step according to an embodiment.

FIG. 4 is a diagram illustrating an example configuration of a device according to an embodiment.

FIGS. 5A through 5E are diagrams illustrating the performance related to runtime and memory usage of a data analytics model according to an embodiment.

FIGS. 6A and 6B are diagrams illustrating the performance related to the accuracy of a data analytics model according to an embodiment.

FIGS. 7A through 7C are diagrams illustrating the performance related to the accuracy of a data analytics model according to an embodiment.

### DESCRIPTION OF THE EMBODIMENTS

The following detailed structural or functional description is provided only for the purpose of providing examples, and various alterations and modifications may be made to the examples. Here, the examples are not construed as limited to the disclosure and should be understood to include all changes, equivalents, and replacements within the idea and the technical scope of the disclosure.

Although terms, such as first, second, and the like are used to describe various components, the components are not limited to the terms. These terms should be used only to distinguish one component from another component. For example, a first component may be referred to as a second component, and similarly the second component may also be referred to as the first component.

It should be noted that, if one component is described as “connected,” “coupled,” or “joined” to another component, a third component may be “connected,” “coupled,” and “joined” between the first and second components, although the first component may be directly connected, coupled, or joined to the second component.

The singular forms “a,” “an,” and “the” are intended to include the plural forms as well, unless the context clearly indicates otherwise. It will be further understood that the terms “comprises/comprising” and/or “includes/including,” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which the present disclosure pertains. Terms, such as those defined in commonly used dictionaries, are to be interpreted as having a meaning that is consistent with their meaning in the context of the relevant art and the disclosure of the present application and is not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like components and a repeated description related thereto will be omitted.

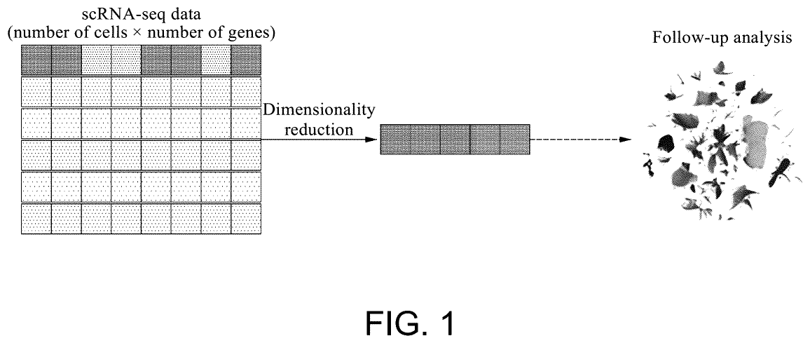

FIG. 1 is a diagram illustrating an example framework of a data analytics model according to an embodiment.

Referring to FIG. 1, a data analytics model according to an embodiment may perform dimensionality reduction for a follow-up analysis of characteristic information. The dimensionality reduction may include a principal component analysis (PCA) of the characteristic information. The characteristic information may include data that is quantitatively measured from a single cell. The characteristic information may include, for example, transcriptomics data, protein data, chromatin accessibility data, or the like. The transcriptomics data may refer to data about a transcript expression level corresponding to each gene in each cell, which may include single-cell RNA sequencing (scRNA-seq) data. The protein data may refer to data about a quantity of a specific protein (e.g., a transcription factor) bound to DNA for each cell. The chromatin accessibility data may refer to cell-specific open DNA sequencing data, which may include data obtained by applying ATAC-seq to a single cell. Hereinafter, the characteristic information will be described with an example of single-cell RNA sequencing data, or scRNA-seq data. This is for illustrative purposes only, and a data analysis method described herein, which is applied to scRNA-seq data, may also be equally applied to any characteristic information quantitatively measurable from a single cell, such as, the protein data or the chromatin accessibility data.

In an embodiment, the scRNA-seq data may include a transcript expression level corresponding to each gene in each cell. For example, the scRNA-seq data may include a matrix in which, with the rows indicating cells and the columns indicating genes (or transcriptomes), a transcript expression level corresponding to each row and column is included as a value. The scRNA-seq data may correspond to a sparse matrix in which most of the values are zero (0) due to a small quantity of transcriptomes in an individual cell and the technical limitations of devices configured to detect transcriptomes inside cells.

In an embodiment, the scRNA-seq data may be count data representing the transcript expression level corresponding to each gene in each cell, corresponding to each of a plurality of batches. Hereinafter, n_{B} denotes the number of batches, and the respective batches may be indexed as b=1, 2, . . . , and n_{B}. Each cell i may be included in a specific batch b, which may be represented as i∈F_{b}. For a cell i∈F_{b}, i may range from 1 to n_{b}, where n_{b} denotes the number of cells included in the batch b. A total number of cells in all the batches may be

$${n}_{C} = {\sum }_{b = 1}^{{n}_{B}} {n}_{b} .$$

The respective genes may be indexed as j=1, 2, . . . , and n_{G}, where n_{G} denotes the number of genes. The entire scRNA-seq data may correspond to a set of matrices in which transcriptomics data, or scRNA-seq data, corresponding to the respective batches is stored. A matrix Y_{b} storing the scRNA-seq data corresponding to the batch b may be represented as Y_{b}∈R^{nb×nG}, and a transcript expression of a gene j of a cell i in the batch b may be represented as Y_{bij}.

Hereinafter, a log-normalization model and a count-based model, which are typical or traditional models implemented to perform dimensionality reduction based on the scRNA-seq data being the count data, will be described.

### Log-Normalization

Since a PCA accounts for a variance present in data in a linear space, data following a normal distribution may be more suitable for the PCA than skewed data. Since scRNA-seq data is count data, the data may be normalized by applying a log-normal transformation. For example, the scRNA-seq data may be normalized by multiplying, by 10000, a value obtained by dividing each Y_{bij} by a total number of transcriptomes in a cell (i.e., a cell size factor), and then applying a log(1+x) function, as expressed in Equation 1 below.

$${\tilde{Y}}_{bij} = log ( 1 + 10000 \times \frac{{Y}_{bij}}{{\sum }_{j ∈ b} {Y}_{bij}} ) \qquad [ Equation 1 ]$$

In Equation 1, other values may be used instead of 10000. For example, in Equation 1, instead of 10000, a positive constant f, with which a mean of a scRNA-seq sum of cells included in a batch b is obtained in a predetermined manner, may be applied. For example, it may be calculated as f=median_{r∈Fb}(m_{bi}). In this example, m_{bi} denotes a cell size factor that is a sum of counts corresponding to all genes in a cell i∈F_{b} or a sum of the total transcript expression level for all the genes, which may be calculated as m_{bi}=ΣᵢY_{bij}.

In general, the PCA may be performed using a singular value decomposition (SVD) algorithm. In a matrix X∈R^{m×n}, X may be represented as a product of three matrices P, Σ, and Q, as shown in Equation 2 below.

$$X = P \sum Q \qquad [ Equation 2 ]$$

In Equation 2, P∈R^{m×k} and Q∈R^{k×n} may have orthonormal columns, and Σ∈R^{k×k} may be a diagonal matrix.

By applying the SVD to a normalized \tilde{Y}_{bij}, PC coordinates or a PC value (e.g., P in Equation 2) of a cell may be calculated. When Y_{bij} is sparse, \tilde{Y}_{bij} may also be sparse. Therefore, even for large data that cannot be stored in a dense matrix, the log-normalization may be easily used when the data is sparse. A disadvantage of the normalization may be, however, that the effects of a small count may be exaggerated, negatively affecting the quality of a downstream analysis.

Count-Based Model (scTransform)

Since scRNA-seq data is inherently count data, the data may be mathematically modeled using a count distribution to avoid artifacts due to normalization. For example, a count-based model may include scTransform, which uses a negative binomial generalized linear model (GLM) along with a conditional mean specification. This scTransform model may include batch covariates (β_{bj}, batches indexed by b), as expressed in Equation 3 below.

$$log ( {μ}_{bij} ) = {β}_{0 j} + {β}_{1 j} log ( {m}_{b i} ) + {\sum }_{b = 2}^{{n}_{B}} {β}_{bj} {X}_{bi} \qquad [ Equation 3 ]$$

Where, μ_{bij} denotes a parameter indicative of a mean estimated using the negative binomial GLM. X_{bi} is an indicator that is 1 if i∈F_{b} and 0 otherwise, and m_{bi} is a cell size factor that is a sum of counts corresponding to all genes in the cell i∈F_{b}, as described above. To obtain the PC coordinates, the SVD may be applied to a Pearson residual obtained from regression. The residual may be expressed in Equation 4 below.

$${r}_{bij} = \frac{{Y}_{bij} - {μ}_{bij}}{\sqrt{{μ}_{bij} + {μ}_{bij}^{2} / {ϕ}_{g}}} \qquad [ Equation 4 ]$$

Where, μ_{bij} and φ_{g} denote mean and variance parameters estimated using the negative binomial GLM. This model may be general and include covariates, but may be slow to be calculated as runtime increases to the square of the number of cells. Also, as the number of batches increases, the number β_{bj} of parameters to fit increases, which may complicate the calculation.

For example, an analytic Pearson residual, which is based on a simpler model, may be used. The analytic Pearson residual may be expressed by considering only a gene-specific intercept β₀ and a cell-specific cell size factor m_{bi}, as shown in Equation 5 below.

$$log ( {μ}_{bij} ) = {β}_{0 j} + log ( {m}_{bi} ) \qquad [ Equation 5 ]$$

According to an embodiment, when a residual matrix (e.g., Pearson residual) is obtained for dimensionality reduction of scRNA-seq data, the residual matrix may correspond to a dense matrix. The residual matrix may be the same as the scRNA-seq data in size, and since the number of cells in the scRNA-seq data is typically very large (e.g., 10⁶⁻⁷), the residual matrix may be a dense matrix with a large size. In this case, storing such a residual matrix may occupy a memory greatly, and thus using the residual matrix to perform a calculation for the dimensionality reduction may slow down the calculation due to a calculation for such a dense matrix.

The data analytics model according to an embodiment may correspond to a model that utilizes a feature that scRNA-seq data is a sparse matrix, applies a probability model, and performs dimensionality reduction by bypassing the residual matrix without directly obtaining it. The data analytics model that performs the dimensionality reduction by bypassing the residual matrix without directly obtaining the residual matrix may be referred to herein as FastRNA. A framework of FastRNA for dimensionality reduction will be described below.

As described above, scRNA-seq data may correspond to count data. In an embodiment, FastRNA is assumed as the same probability model as Equation 3 where a mean in a count distribution of the scRNA-seq data is specified by a cell size factor m_{bi} and a batch covariate β_{bj} (batch indexed by b). It is also assumed to follow a Poisson distribution where counts of the scRNA-seq data have a specified mean. For example, the data analytics model may be expressed as Equation 6 below, where

$${β}_{0 j} + {\sum }_{b = 2}^{{n}_{B}} {β}_{bj} {X}_{bi}$$

in Equation 3 is replaced with a new parameter, B_{bj}.

$$log ( {μ}_{bij} ) = {B}_{bj} + log ( {m}_{b i} ) for i ∈ {Φ}_{b} \qquad [ Equation 6 ]$$

A model proposed by Equation 6 above may differ from scTransform in that it applies the Poisson distribution instead of the negative binomial distribution.

According to an embodiment, a Pearson residual r_{bij} may be expressed as shown in Equation 7 below.

$${r}_{bij} = \frac{{Y}_{bij} - {μ}_{bij}}{{σ}_{bij}} \qquad [ Equation 7 ]$$

Since the Poisson distribution has a relationship s²_{bij}=μ_{bij}, a value of μ_{bij} may need to be estimated to calculate r_{bij}. In general, estimating μ_{bij} requires estimating B_{bj} by performing a full regression on Y_{bij}. A method to bypass a direct estimation of B_{bj} to improve efficiency using a sufficient statistic will be described below.

The sufficient statistic may be defined as follows. For a probability distribution f_{θ,y}(x) with a parameter θ (where v is a nuisance parameter other than θ) and data X, a sufficient statistic T(X) for θ may be a summary of the data X that includes all the information related to the estimation of θ. Thus, given (or conditional on) T(X), the distribution of X may be independent of θ.

According to an embodiment, it may be observed that Σ_{i∈Fb}Y_{bij} is a sufficient statistic for B_{bj}. That is, conditionally on Σ_{i∈Fb}Y_{bij}, Y_{bij} may no longer be dependent on B_{bj}. This may be derived by deriving the conditional distribution of Y_{bij}, which follows a multinomial distribution whose parameters are independent of B_{bj}. A detailed process may be as shown in Equation 9 below. In this case, a resulting conditional distribution may be specified as shown in Equation 8 below.

$${Y}_{bij} \vert {\sum }_{i ∈ {Φ}_{b}} {Y}_{bij} ∼ Mult ( {\sum }_{i ∈ {Φ}_{b}} {Y}_{bij} , \{ {π}_{bij} \} ) , \qquad [ Equation 8 ]$$

$${π}_{bij} = \frac{{e}^{{B}_{bj} + log {m}_{bi}}}{{\sum }_{{i}^{′} ∈ {Φ}_{b}} {e}^{{B}_{bj} + log {m}_{{bi}^{′}}}} = \frac{{m}_{bi}}{{\sum }_{{i}^{′} ∈ {Φ}_{b}} {m}_{{bi}^{′}}}$$

$$\begin{matrix}P ( {Y}_{b {1}_{\text{?}}} , … , {Y}_{{bn}_{\text{?}}} \vert {\sum }_{i ∈ {Φ}_{b}} {Y}_{bij} ) = \frac{P ( {Y}_{\text{?}} , … , {Y}_{\text{?}} {\sum }_{\text{?}} {Y}_{bij} )}{P ( {\sum }_{\text{?}} {\text{?}}_{\text{?}} )} \\ = \frac{P ( {Y}_{\text{?}} , … , {Y}_{\text{?}} )}{P ( {\sum }_{\text{?}} {\text{?}}_{\text{?}} )} \\ = \frac{{\prod }_{\text{?}} P ( {Y}_{\text{?}} )}{P ( {\sum }_{\text{?}} {\text{?}}_{\text{?}} )} \\ = \frac{{\prod }_{\text{?}} \frac{\text{?}}{\text{?}}}{\frac{\text{?}}{\text{?}}} \\ = \frac{\text{?}}{\text{?}} \cdot {\prod }_{\text{?}} {( \frac{\text{?}}{\text{?}} )}^{\text{?}} \\ = \frac{\text{?}}{\text{?}} \cdot {\prod }_{\text{?}} {( \frac{\text{?}}{\text{?}} )}^{\text{?}} \\ = \frac{\text{?}}{\text{?}} \cdot {\prod }_{\text{?}} {( \frac{\text{?}}{\text{?}} )}^{\text{?}}\end{matrix} \qquad [ Equation 9 ]$$

$$\text{?} \text{indicates text missing or illegible when filed}$$

Based on this observation, under the assumption of FastRNA being a conditional Poisson distribution, rather than a marginal Poisson distribution, a strategy using a Pearson residual may be set. The rationale behind this may be as follows. A conditional on Σ_{i∈Fb}Y_{bij} may be considered to have a similar effect to estimating and regressing B_{bj} in the sense that it removes the effects of B_{bj} on Y_{bij}. The intuition behind the residual calculation may be to calculate a deviation of data from a model, under the assumption of a parametric null model, and thus whether to use the marginal or conditional distribution may be a subjective choice. Choosing the conditional Poisson distribution may achieve equivalent performance to choosing the marginal Poisson distribution, while having the following advantages. First, estimating each B_{bj} through regression may not be required, and thus the complexity of an algorithm that calculates r_{bij} using Equation 8 may be insensitive to the number of batches. Second, the mean and variance of the multinomial distribution may be easily calculated, which may simplify the calculation of residuals. The conditional expectation and the conditional variance may be calculated as expressed in Equation 10.

$${μ}_{bij} = 𝔼 [ {Y}_{bij} \vert {\sum }_{i ∈ {Φ}_{b}} {Y}_{bij} , log {m}_{bi} ] = [ {\sum }_{i ∈ {Φ}_{b}} {Y}_{bij} ] \cdot {π}_{bij} \qquad [ Equation 10 ]$$

$${σ}_{bij}^{2} = Var [ {Y}_{bij} \vert {\sum }_{i ∈ {Φ}_{b}} {Y}_{bij} , log {m}_{bi} ] = [ {\sum }_{i ∈ {Φ}_{b}} {Y}_{bij} ] \cdot {π}_{bij} ( 1 - {π}_{bij} )$$

Where, using Equation 10 may enable efficient generation of the Pearson residual R={r_{bij}}. The number of rows and columns of R may be the number of genes and the number of cells, respectively.

Since R is a dense matrix, the computational complexity of PCA using R may still be large. A method without direct use of R for PCA will be described below.

According to an embodiment, two approaches may be used to perform PCA on R. One is to apply an SVD to R, and the other is to apply eigen-decomposition to a covariance matrix RR^{T} of R. Both approaches may provide the same result, and thus one of the two may be used to perform PCA.

According to an embodiment, instead of applying SVD directly to R, a strategy of applying eigen-decomposition to RR^{T} may be used. For a large scRNA-seq dataset, the number of genes is much smaller than the number of cells, and thus RR^{T} is a small matrix of size (number of genes)×(number of genes). In general, for scRNA-seq data, RR^{T} may be easily stored in a memory of less than 1 GB.

According to an embodiment, by decomposing RR^{T} into three elements, RR^{T} may be calculated efficiently, and the three elements included in RR^{T} may be calculated by sparse matrix algebra. Under the assumption that A={Y_{bij}/σ_{bij}} and B={μ_{bij}/σ_{bij}}, A may be a sparse matrix and B may be essentially a repetition of the same vector. RR^{T} may be expressed as shown in Equation 11 below.

$$\begin{matrix}{RR}^{T} = ( A - B ) {( A - B )}^{T} \\ = {AA}^{R} - 2 {AB}^{T} + {BB}^{T}\end{matrix} \qquad [ Equation 11 ]$$

The three terms in Equation 11 may be calculated in a sparse manner for the same reason as in Equation 12 below.

$${n}_{bi} = {\sum }_{j} {Y}_{bij} , {n}_{bj} = {\sum }_{i ∈ {Φ}_{b}} {Y}_{bij} , {n}_{b} = {\sum }_{i ∈ {Φ}_{b , j}} {Y}_{bij} \\ {p}_{bi} = {n}_{bi} / {n}_{b} . \\ \begin{matrix}{AB}_{j {j}^{′}}^{T} = {\sum }_{b = 1}^{{n}_{B}} {\sum }_{k ∈ {Φ}_{b}} {A}_{b kj} {B}_{{bkj}^{′}} \\ = {\sum }_{b = 1}^{{n}_{B}} {\sum }_{k ∈ {Φ}_{b}} {A}_{bkj} \sqrt{{n}_{{bj}^{′}} \frac{{p}_{bk}}{1 - {p}_{bk}}} \\ = {\sum }_{b = 1}^{{n}_{B}} \sqrt{{n}_{{bj}^{′}}} {\sum }_{k ∈ {Φ}_{b}} {A}_{bkj} \sqrt{\frac{{p}_{bk}}{1 - {p}_{bk}}} \\ {BB}_{j {j}^{′}}^{T} = {\sum }_{b = 1}^{{n}_{B}} \sqrt{{n}_{bj} {n}_{{bj}^{′}}} {\sum }_{k ∈ {Φ}_{b}} \frac{{p}_{bk}}{1 - {p}_{bk}}\end{matrix} \qquad [ Equation 12 ]$$

Therefore, RR^{T} may be calculated very efficiently by using the sparsity of scRNA-seq data. The dense matrix R may not need to be stored in a memory to calculate RR^{T}, and since R itself is not calculated, there may be no need to store R in the memory, which may reduce memory requirements.

For a matrix X as in Equation 2, P is a projection of X onto an eigen-space of X¹¹. Also, an eigen-space of X may be the same as an eigen-space of XX^{T}. By applying eigen-decomposition to RR^{T}, a matrix, U∈M^{D×J}, of eigenvectors spanning the eigen-space of R may be obtained. R may be projected onto a column space of U. That is, PC coordinates of a cell, P=UR, may need to be calculated. This projection may be obtained using only sparse algebra, as expressed in Equation 13.

$$\begin{matrix}U {R}_{bid} = U {A}_{bid} - U {B}_{bid} \\ = U {A}_{bid} - {\sum }_{j} {U}_{dj} {B}_{bij} \\ = U {A}_{bid} - {\sum }_{j} {U}_{dj} \sqrt{{n}_{bj} \frac{{p}_{bi}}{1 - {p}_{bi}}} \\ = U {A}_{bid} - \sqrt{\frac{{p}_{bi}}{1 - {p}_{bi}}} {\sum }_{j} {U}_{dj} \sqrt{{n}_{bj}}\end{matrix} \qquad [ Equation 13 ]$$

Where, UR_{bid} denotes a dth PC value of cell i in batch b.

According to an embodiment, in an analysis of scRNA-seq data, PCA may be generally performed using a subset of significant features, instead of using all features (or genes). For example, a specific number (e.g., 1,000) of genes with the largest variation may be selected, and PCA may be performed on a subset including the selected genes. In a similar way to the PCA calculation method described above, a calculation for feature selection may be performed efficiently using sparse algebra.

A variance of residuals may be calculated as an operation of a sparse matrix, as expressed in Equation 14 below.

$${\hat{V}}_{j} = \frac{1}{{\sum }_{b} {I}_{b}} {\sum }_{b} {\sum }_{i ∈ b} {r}_{bij}^{2} \\ {r}_{bij}^{2} = \frac{{Y}_{bij}^{2}}{{σ}_{bij}^{2}} - 2 \frac{{Y}_{bij} {μ}_{bij}}{{σ}_{bij}^{2}} + \frac{{μ}_{bij}^{2}}{{σ}_{bij}^{2}} \\ \frac{{Y}_{bij} {μ}_{bij}}{{σ}_{bij}^{2}} = \frac{{Y}_{bij}}{1 - {π}_{bij}} \\ \begin{matrix}\frac{{μ}_{bij}^{2}}{{σ}_{bij}^{2}} = \frac{{μ}_{bij}}{1 - {π}_{bij}} \\ = [ {\sum }_{{i}^{′} ∈ b} {Y}_{{bi}^{′} j} ] \cdot \frac{{π}_{bij}}{1 - {π}_{bij}}\end{matrix} \qquad [ Equation 14 ]$$

Therefore, the operation of selecting a subset of features to be used for PCA may be performed with high time and memory efficiency.

In summary, an important characteristic of the entire pipeline for data analysis in FastRNA is that the dense matrix R is not calculated or stored in a memory. In addition to allowing control for batch effects, FastRNA may provide a basis for applying a unique algebraic strategy to avoid the need to calculate R for both PCA and feature selection. In fact, according to an embodiment, the PCA calculation performed in FastRNA may require several times less time and memory than a traditional or typical PCA, and the calculation for feature selection may also require less than one-tenth the time and memory compared to a traditional or typical one for feature selection. In addition, FastRNA does not approximate the calculation, and may thus provide a solution for an accurate PCA calculation without sacrificing the precision of the calculation.

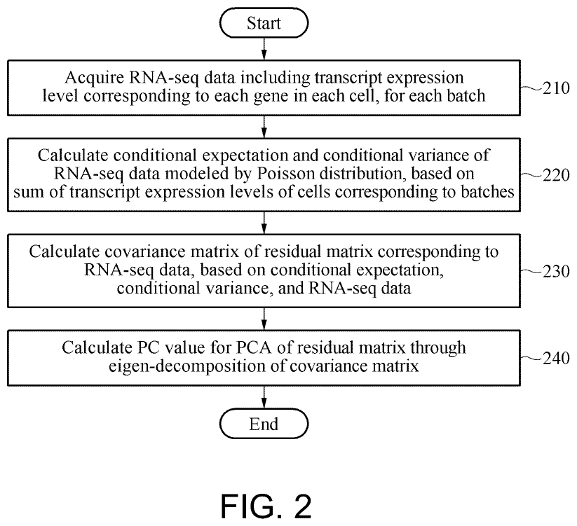

FIG. 2 is a flowchart illustrating a flow of steps of a data analysis method according to an embodiment.

Referring to FIG. 2, a data analysis method according to an embodiment may include: step 210 of acquire characteristic information quantitatively measured from a single cell, corresponding to each of a plurality of batches; step 220 of calculating a conditional expectation and a conditional variance of the characteristic information modeled to a Poisson distribution based on a sum of the characteristic information of cells corresponding to each of the plurality of batches; step 230 of calculating a covariance matrix of a residual matrix corresponding to the characteristic information, based on the conditional expectation, the conditional variance, and the characteristic information; and step 240 of calculating a PC value for a PCA of the residual matrix by performing eigen-decomposition on the covariance matrix. In an embodiment, the data analysis method may correspond to operations performed by FastRNA described above. That is, it may correspond to an operation of FastRNA performed for dimensionality reduction for a follow-up analysis of characteristic information (e.g., RNA sequencing data) quantitatively measured from a single cell, which has been described above with reference to FIG. 1.

As described above, the characteristic information quantitatively measured from a single cell, which is acquired in step 110, may include at least one of the following: transcriptomics data (e.g., RNA sequencing data, or scRNA-seq data) including a transcript expression level corresponding to each gene in each cell, data on a quantity of certain protein (e.g., transcription factors) bound to DNA for each cell, and open DNA sequencing data for each cell. Hereinafter, the characteristic information will be described, with the transcriptomics data as an example.

For example, the transcriptomics data may include transcriptomics data of each cell, and may include, for example, transcript expression levels corresponding to 20,000 genes in 10⁷ cells. The transcriptomics data may correspond to Y_{bij} described above. That is, the transcriptomics data may include count data of a jth gene of cell i in batch b. As described above, the transcriptomics data may correspond to a sparse matrix where most of the values are zero (0).

According to an embodiment, a batch may correspond to a unit of processing the transcriptomics data. The transcriptomics data may include transcriptomics data of each of a plurality of cells, which may be divided into batch units. For example, a batch may correspond to a donor of cells, and transcriptomic data corresponding to cells from the same donor may be divided into a single batch unit.

In an embodiment, the characteristic information modeled to the Poisson distribution in step 220 may correspond to the characteristic information (e.g., transcriptomics data) modeled by Equation 6 above. A sum of the characteristic information of the cells corresponding to each of the plurality of batches may correspond to Σ_{i∈Fb}Y_{bij-} as described above.

In an embodiment, step 220 of calculating the conditional expectation and the conditional variance may include modeling the characteristic information to a multinomial distribution conditional on the sum of the characteristic information of the cells corresponding to each of the plurality of batches, and calculating the conditional expectation and the conditional variance according to the multinomial distribution of the characteristic information.

According to an embodiment, the characteristic information may be modeled to follow the multinomial distribution conditional on Σ_{i∈Fb}Y_{bij}, as shown in Equation 8. Based on the multinomial distribution, the conditional expectation μ_{bij} and the conditional variance σ²_{bij} of the characteristic information may be calculated as expressed in Equation 10.

In an embodiment, in step 230, the residual matrix may correspond to R described above. Referring to Equation 7, the residual matrix may be equivalent to a matrix A-B obtained by subtracting B from A, where A={Y_{bij}/σ_{bij}) and B={μ_{bij}/σ_{bij}}. The covariance matrix of the residual matrix may be equivalent to RR^{T}, which is a product of the residual matrix R and a transposed matrix RT of the residual matrix. Therefore, as shown in Equation 11, the covariance matrix of the residual matrix may correspond to (A−B)(A−B)^{T}, which may be obtained by calculating AA^{T}+2AB^{T}+BB^{T} deployed from (A−B)(A−B)^{T}. That is, the step of calculating the covariance matrix of the residual matrix may include calculating a sum of AA^{T}, 2AB^{T}, and BB^{T}.

Since Y_{bij} is a sparse matrix, A may also be a sparse matrix. AA^{T} may be calculated by a multiply operation of a sparse matrix. For example, AB^{T} and BB^{T} may be calculated as expressed in Equation 12. Referring to Equation 12, AB^{T} may be calculated as

$${\sum }_{b = 1}^{{n}_{B}} \sqrt{{n}_{{bj}^{′}}} {\sum }_{k ∈ {Φ}_{b}} {A}_{bkj} \sqrt{\frac{{p}_{bk}}{1 - {p}_{bk}}} ,$$

where

$${A}_{bkj} \sqrt{\frac{{p}_{bk}}{1 - {p}_{bk}}}$$

is a product of a sparse matrix and a vector, and a result of the operation may be a vector. Since \sqrt{nb_{j′}} is a vector, AB^{T} may be calculated by a sparse matrix-and-vector operation and a vector operation. Since BB^{T} is

$${\sum }_{b = 1}^{{n}_{B}} \sqrt{{n}_{bj} {n}_{{bj}^{′}}} {\sum }_{k ∈ {Φ}_{b}} \frac{{p}_{bk}}{1 - {p}_{bk}} ,$$

it may be calculated by a vector operation. That is, the covariance matrix of the residual matrix may be calculated as a sum of terms obtained by the sparse matrix-and-vector operation or the vector operation.

In an embodiment, step 240 may include obtaining an eigenvector corresponding to an eigen-space of the residual matrix by performing eigen-decomposition on the covariance matrix of the residual matrix, and calculating the PC value of the residual matrix that is a projection of the residual matrix onto the eigen-space, based on the eigenvector.

In an embodiment, the eigenvector may correspond to U described above. The PC value of the residual matrix may be calculated according to Equation 13. Referring to Equation 13, the PC value of the residual matrix may be calculated as

$$U {A}_{bid} - \sqrt{\frac{{p}_{bi}}{1 - {p}_{bi}}} {\sum }_{j} {U}_{dj} \sqrt{{n}_{bj}} .$$

Since A is a sparse matrix, and U is not a sparse matrix but a small-sized matrix, the PC value of the residual matrix may be calculated by an operation between the small-sized matrix and a vector and an operation between the small-sized matrix and the sparse matrix.

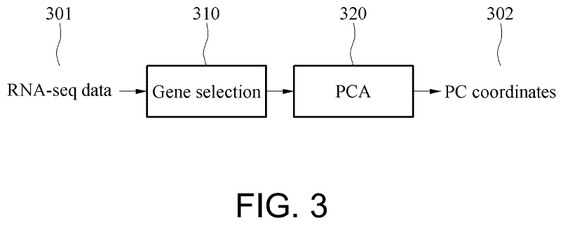

FIG. 3 is a diagram illustrating a data analysis method including a gene selection step according to an embodiment.

Referring to FIG. 3, the data analysis method according to an embodiment may further include gene selection step 310 before PCA step 320. For example, the PCA step 320 may include step 230 and step 240 described above with reference to FIG. 2.

According to an embodiment, the PCA step 320 for characteristic information 301 (e.g., transcriptomics data) may be performed on a subset of the characteristic information 301 including some genes, instead of using all genes. For example, a specific number (e.g., 1,000) of genes with the largest variation may be selected, and the PCA step 320 may be performed on a subset including the selected genes. In this case, a variation for selecting genes may be measured by a variance of residuals of characteristic information.

According to an embodiment, the data analysis method may include a step of calculating a variance of residuals corresponding to each gene in characteristic information, based on a conditional expectation, a conditional variance, and the characteristic information; determining a subset of the characteristic information including at least some of genes included in the characteristic information based on the variance of the residuals corresponding to each gene; and calculating a covariance matrix of a residual matrix corresponding to the determined subset. The conditional expectation and the conditional variance may correspond to the conditional expectation and the conditional variance based on the multinomial distribution of the characteristic information, which are calculated in step 220 described above with reference to FIG. 2.

In an embodiment, the variance of the residuals may correspond to a variance of residuals calculated according to Equation 14 above. Referring to Equation 14, r²_{bij} may be represented as

$$\frac{{Y}_{bij}^{2}}{{σ}_{bij}^{2}} - 2 \frac{{Y}_{bij} {μ}_{bij}}{{σ}_{bij}^{2}} + \frac{{μ}_{bij}^{2}}{{σ}_{bij}^{2}} . \\ \frac{{Y}_{bij}^{2}}{{σ}_{bij}^{2}}$$

may be calculated by a multiply operation of a sparse matrix.

$$\frac{{Y}_{bij} {μ}_{bij}}{{σ}_{bij}^{2}}$$

may be equivalent to

$$\frac{{Y}_{bij}}{1 - {π}_{bij}} ,$$

and may correspond to the sparse matrix.

$$\frac{{μ}_{bij}^{2}}{{σ}_{bij}^{2}}$$

may be equivalent to

$$[ {\sum }_{{i}^{′} ∈ b} {Y}_{{bi}^{′} j} ] \cdot \frac{{π}_{bij}}{1 - {π}_{bij}} ,$$

and may be calculated by a sparse matrix-and-vector operation.

In an embodiment, the step of determining the subset of the characteristic information may include selecting a predetermined number of genes in an order of increasing variance of the residuals and determining the subset of the characteristic information including the selected genes. For example, top n genes (e.g., n=1000) having a large variance of residuals may be selected, and a subset of characteristic information including columns corresponding to the selected genes in the characteristic information may be determined. That is, the columns of the subset of characteristic information may correspond to the selected 1000 genes.

According to an embodiment, the PCA step 320 may be performed on the subset of the characteristic information, and PC coordinates 302 may be output as a result of performing the PCA step 320.

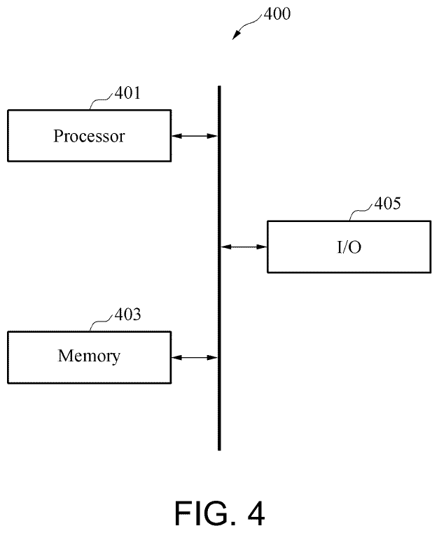

FIG. 4 is a diagram illustrating an example configuration of a device according to an embodiment.

Referring to FIG. 4, a device 400 according to an embodiment may include a processor 401, a memory 403, and an input/output (I/O) device 405. In an embodiment, the device 400 may correspond to a device on which the data analytics model described above with reference to FIGS. 1 through 3 is implemented.

In an embodiment, the processor 401 may perform at least one of the operations or steps of the data analysis method described above with reference to FIGS. 1 through 3. For example, the processor 401 may perform at least one of the following steps: obtaining characteristic information quantitatively measured from a single cell, corresponding to each of a plurality of batches; calculating a conditional expectation and a conditional variance of the characteristic information modeled to a Poisson distribution, based on a sum of the characteristic information of cells corresponding to each of the plurality of batches; calculating a covariance matrix of a residual matrix corresponding to the characteristic information, based on the conditional expectation, the conditional variance, and the characteristic information; and calculating a PC value for PCA of the residual matrix by performing eigen-decomposition on the covariance matrix.

In an embodiment, the memory 403 may be a volatile memory or a non-volatile memory and may store data related to the data analysis method described above with reference to FIGS. 1 through 3. For example, the memory 403 may store data generated in the process of performing the data analysis method or data required to perform the data analysis method.

The device 400 according to an embodiment may be connected to an external device (e.g., a personal computer (PC) or a network) through the I/O device 405 and may exchange data. For example, the device 400 may receive characteristic information as input data through the I/O device 405, and may output a PC value calculated as a result of performing PCA on the characteristic information.

In an embodiment, the memory 403 may store a program in which the data analysis method described above with reference to FIGS. 1 through 3 is implemented. The processor 401 may execute the program stored in the memory 403 and control the device 400. Code of the program executed by the processor 401 may be stored in the memory 403.

The device 400 according to an embodiment may further include other components not shown. For example, the device 400 may provide functionality for the device 400 to communicate with other electronic devices or other servers over a network. That is, the device 400 may further include a communication module for communicating with other devices. For example, the device 400 may further include other components such as a transceiver, various sensors, a database (DB), or the like.

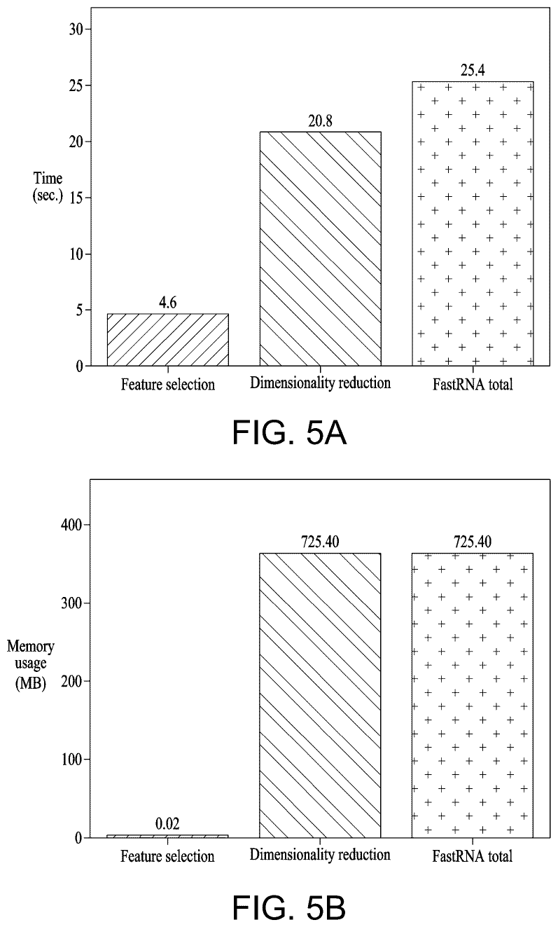

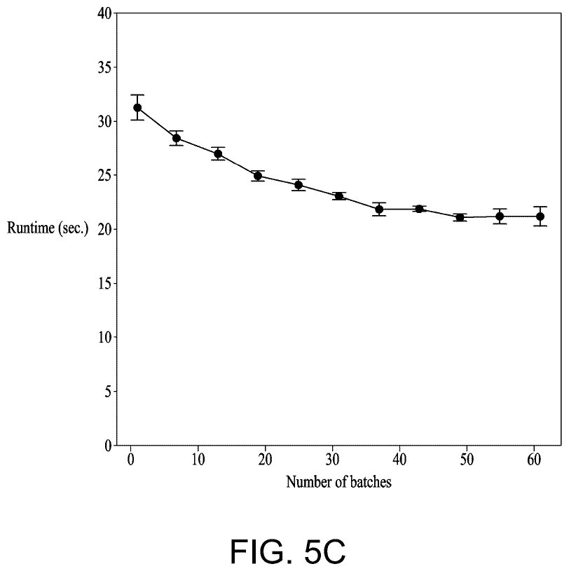

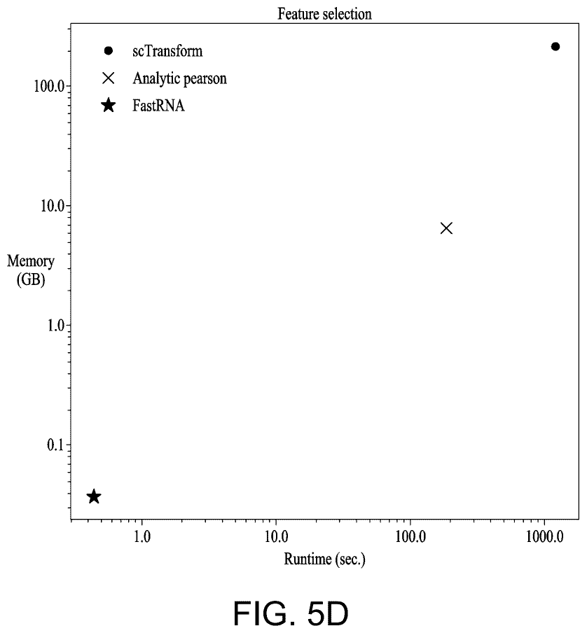

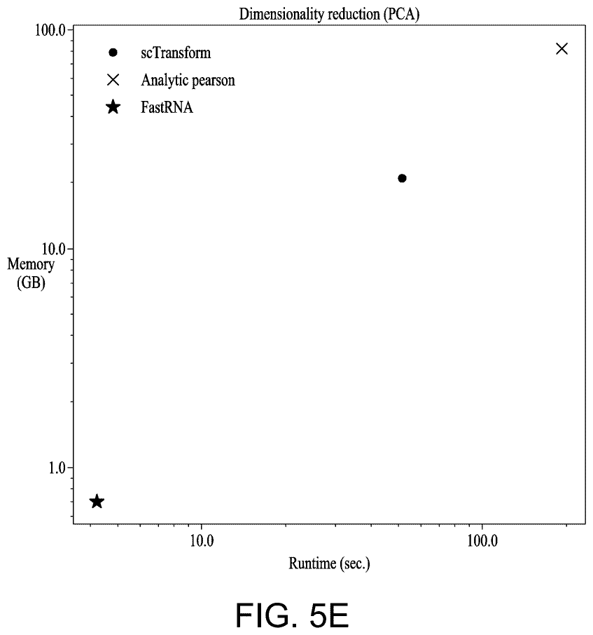

FIGS. 5A through 5E are diagrams illustrating the performance related to runtime and memory usage of a data analytics model according to an embodiment.

Referring to FIGS. 5A and 5B, shown are results of benchmarking the runtime and memory efficiency of a data analytics model or FastRNA according to an embodiment. For this purpose, FastRNA was used to perform dimensionality reduction (e.g., PCA) on an atlas-scale mouse dataset including 2 million cells. This dataset includes 62 batches. Referring to FIG. 5A, a result of measuring runtime in a benchmarking environment showed that 4.6 seconds was used to complete feature selection and 20.8 seconds was used to complete dimensionality reduction, and accordingly 25 seconds was used to complete dimensionality reduction of the entire dataset (FastRNA total). Referring to FIG. 5B, a result of measuring memory usage in the benchmarking environment showed that 0.02 MB was used to complete feature selection and 725.40 MB was used to complete dimensionality reduction, and accordingly 725 MB of memory was used to complete dimensionality reduction of the entire dataset (fastRNA total requirement). Using a traditional or typical method (e.g., scTransform, analytic Pearson residuals), performing dimensionality reduction on the same data may require more than 100 GB of memory and hours of runtime, which may demonstrate that using FastRNA significantly improves the memory usage and runtime.

Referring to FIG. 5C, shown is a result of measuring the runtime of FastRNA depending on the number of batches. To measure the effect or influence of the number of batches on the runtime of FastRNA, the runtime was measured with the number of batches gradually increasing from 1 to 62 while the batches were randomly assigned in disregard of actual batch labels in an input dataset. Unlike a traditional method in which the runtime varies sensitively to the number of batches, the runtime of FastRNA was not affected by the number of batches. In fact, the result showed that, as the number of batches increases, the runtime decreases slightly.

Referring to FIG. 5D and FIG. 5E, shown are results of comparing FastRNA to scTransform and analytic Pearson residuals, which are traditional count-based methods, in terms of efficiency. The time and memory required to perform PCA for FastRNA, scTransform, and analytic Pearson residuals, respectively, were measured using a dataset obtained by downsampling an atlas-scale mouse dataset to 200,000 cells. The results showed that FastRNA requires 30 MB of memory and 4 seconds to process the 200,000 cells. The results showed that, on the other hand, scTransform requires 216 GB of memory and 6424 seconds of time, and the analytic Pearson residuals requires 83 GB of memory and 382 seconds of time. It is therefore found that the time requirement of FastRNA is more than 100 times smaller than the other methods, and the memory requirement of FastRNA is more than 1000 times smaller than the other methods.

FIGS. A and 6B are diagrams illustrating the performance related to the accuracy of a data analytics model according to an embodiment.

To check the accuracy of a result of performing PCA, whether cells of the same type are clustered together in a PC space may be determined. For this purpose, the accuracy of results of performing FastRNA, scTransform, analytic Pearson residuals, and log-normalization (LogNorm) on a dataset specified as a fluorescence-activated cell sorting (FACS) label was measured. FastRNA, scTransform, and analytic Pearson residuals are count-based models. On the other hand, the LogNorm model is a non-count-based model.

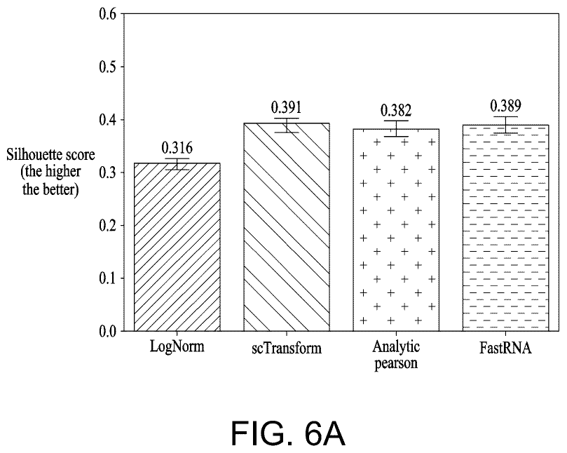

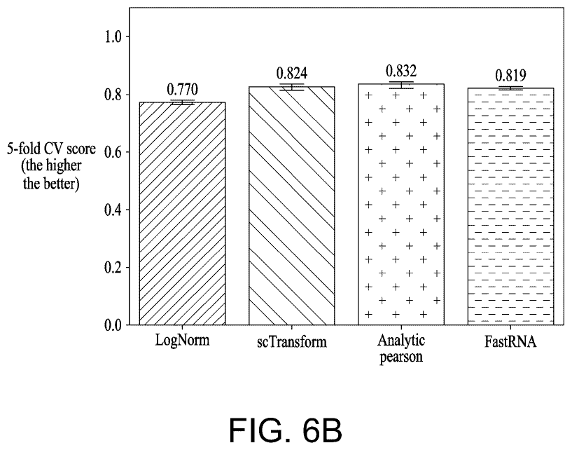

Referring to FIG. 6A, shown is a result of measuring silhouette scores of the results of FastRNA, scTransform, analytic Pearson residuals, and LogNorm. A silhouette score is a measure of how well PC coordinates reflect a label of cells. The higher the silhouette score, the better the PC coordinates reflect a label of cells. The result showed that the silhouette score (0.389) of FastRNA is higher than the silhouette score (0.316) of LogNorm, and is similar to the respective silhouette scores (0.391 and 0.382) of scTransform and analytic Pearson residuals which are count-based.

Referring to FIG. 6B, shown is a result of measuring 5-fold CV scores of the results of FastRNA, scTransform, analytic Pearson residuals, and LogNorm. A 5-fold CV score is a measure of how well five nearest neighbors of a point predict the cell type classification of that point, given that the point is in a test set with 20% occupied and the neighbors are in a training set with 80% occupied. The result showed that the 5-fold CV score (0.819) of FastRNA is higher than the 5-fold CV score (0.770) of LogNorm, and is similar to the respective 5-fold CV scores (0.824 and 0.832) of scTransform and analytic Pearson residuals which are count-based.

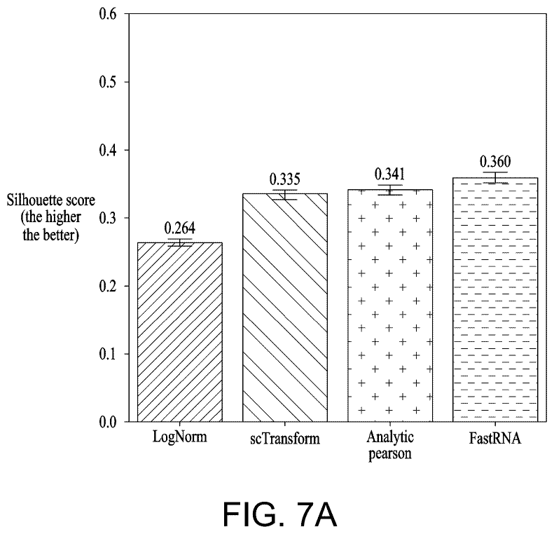

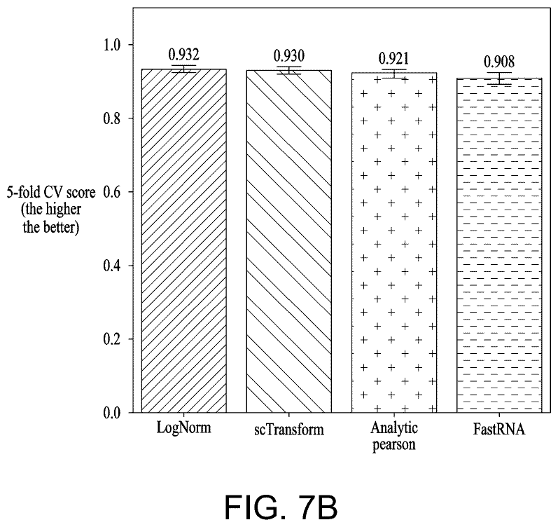

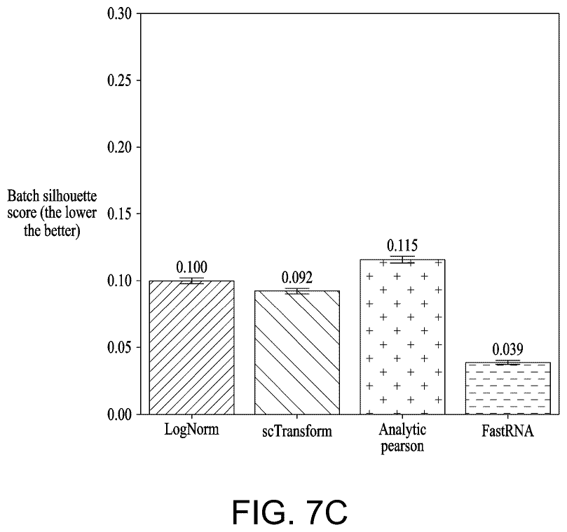

FIGS. 7A through 7C are diagrams illustrating the performance related to the accuracy of a data analytics model according to an embodiment.

Using a peripheral blood mononuclear cell (PBMC) dataset generated from three 10X experiments, the accuracy of the data analytics model, when there is a plurality of batches in a single dataset, was measured. The dataset is a mixture of data generated by three different versions of generation technology (e.g., 10X V2A, 10X V2B, and 10X V3). Each of the versions may have its own bias, and thus the generation technology may be considered to have batch effects.

Referring to FIG. 7A, shown is a result of measuring silhouette scores of the results from FastRNA, scTransform, analytic Pearson residuals, and LogNorm. The result showed that the silhouette score (0.360) of FastRNA is higher than the silhouette score (0.264) of LogNorm, and is similar to the respective silhouette scores (0.335 and 0.341) of scTransform and analytic Pearson residuals which are count-based.

Referring to FIG. 7B, shown is a result of measuring 5-fold CV scores of the results of FastRNA, scTransform, analytic Pearson residuals, and LogNorm. The result showed that the 5-fold CV score (0.908) of FastRNA is similar to the 5-fold CV scores of the other models.

Referring to FIG. 7C, shown is a result of calculating batch silhouette scores of the results of FastRNA, scTransform, analytic Pearson residuals, and LogNorm. Since clustering by batch is not desired by cells, a lower silhouette score may indicate that PC coordinates better reflect a label of cells. The result showed that the batch silhouette score (0.039) of FastRNA is less than half of the batch silhouette score (0.092) of scTransform which is the second lowest scoring method.

The embodiments described herein may be implemented using hardware components, software components and/or combinations thereof. A processing device may be implemented using one or more general-purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such as, parallel processors.

The software may include a computer program, a piece of code, an instruction, or some combination thereof, to independently or collectively instruct or configure the processing device to operate as desired. The software and/or data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be those specially designed and constructed for the purposes of examples, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described hardware devices may be configured to act as one or more software modules in order to perform the operations of the above-described examples, or vice versa.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above disclosure, the scope of the disclosure may also be defined by the claims and their equivalents, and all variations within the scope of the claims and their equivalents are to be construed as being included in the disclosure.

1. A data analysis method, comprising:
acquiring characteristic information quantitatively measured from a single cell, corresponding to each of a plurality of batches;
calculating a conditional expectation and a conditional variance of the characteristic information modeled to a Poisson distribution, based on a sum of the characteristic information of cells corresponding to each of the plurality of batches;
calculating a covariance matrix of a residual matrix corresponding to the characteristic information, based on the conditional expectation, the conditional variance, and the characteristic information; and
calculating a PC value for a principal component analysis (PCA) of the residual matrix through eigen-decomposition of the covariance matrix.

2. The data analysis method of claim 1, wherein the calculating of the conditional expectation and the conditional variance comprises:
modeling the characteristic information to a multinomial distribution conditional on the sum of the characteristic information of the cells corresponding to each of the plurality of batches; and
calculating the conditional expectation and the conditional variance based on the multinomial distribution of the characteristic information.

3. The data analysis method of claim 1, wherein the calculating of the covariance matrix of the residual matrix comprises:
calculating a sum of AA^{T}, 2AB^{T}, and BB^{T},
wherein A denotes Y_{bij}/σ_{bij}; B denotes μ_{bij}/σ_{bij}; Y_{bij} denotes characteristic information corresponding to batch b, cell i, and gene j; μ_{bij} denotes a conditional expectation of Y_{bij}; and σ_{bij} denotes a conditional variance of Y_{bij}.

4. The data analysis method of claim 3, wherein the residual matrix is equivalent to a matrix obtained by subtracting B from A.

5. The data analysis method of claim 1, wherein the covariance matrix of the residual matrix is equivalent to a product of the residual matrix and a transposed matrix of the residual matrix.

6. The data analysis method of claim 3, wherein AB^{T} and BB^{T} are calculated as expressed in the following equation,
$${n}_{bi} = {\sum }_{j} {Y}_{bij} , {n}_{bj} = {\sum }_{i ∈ {Φ}_{b}} {Y}_{bij} , {n}_{b} = {\sum }_{i ∈ {Φ}_{b , j}} {Y}_{bij} \\ {p}_{bi} = {n}_{bi} / {n}_{b} . \\ \begin{matrix}{AB}_{j {j}^{′}}^{T} = {\sum }_{b = 1}^{{n}_{B}} {\sum }_{k ∈ {Φ}_{b}} {A}_{bkj} {B}_{{bkj}^{′}} \\ = {\sum }_{b = 1}^{{n}_{B}} {\sum }_{k ∈ {Φ}_{b}} {A}_{bkj} \sqrt{{n}_{{bj}^{′}} \frac{{p}_{bk}}{1 - {p}_{bk}}} \\ = {\sum }_{b = 1}^{{n}_{B}} \sqrt{{n}_{{bj}^{′}}} {\sum }_{k ∈ {Φ}_{b}} {A}_{bkj} \sqrt{\frac{{p}_{bk}}{1 - {p}_{bk}}} \\ {BB}_{j {j}^{′}}^{T} = {\sum }_{b = 1}^{{n}_{B}} \sqrt{{n}_{bj} {n}_{{bj}^{′}}} {\sum }_{k ∈ {Φ}_{b}} \frac{{p}_{bk}}{1 - {p}_{bk}}\end{matrix} \qquad [ Equation ]$$

7. The data analysis method of claim 1, wherein the calculating of the covariance matrix of the residual matrix comprises:
calculating a variance of a residual corresponding to each gene in the characteristic information, based on the conditional expectation, the conditional variance, and the characteristic information;
determining a subset of the characteristic information comprising at least a portion of genes comprised in the characteristic information, based on the variance of the residual corresponding to each gene; and
calculating a covariance matrix of a residual matrix corresponding to the determined subset.

8. The data analysis method of claim 7, wherein the determining of the subset of the characteristic information comprises:
selecting a predetermined number of genes in an order of increasing variance of the residual; and
determining the subset of the characteristic information comprising the selected genes.

9. The data analysis method of claim 1, wherein the calculating of the PC value of the residual matrix comprises:
acquiring an eigenvector corresponding to an eigen-space of the residual matrix by performing eigen-decomposition on the covariance matrix of the residual matrix; and
calculating the PC value of the residual matrix, which is a projection of the residual matrix onto the eigen-space, based on the eigenvector.

10. The data analysis method of claim 1, wherein the characteristic information comprises at least one of:
RNA sequencing data comprising a transcript expression level corresponding to each gene in each cell;
data on a quantity of transcription factors bound to DNA for each cell; and
open DNA sequencing data for each cell.

11. The data analysis method of claim 1, wherein the characteristic information is a sparse matrix.

12. The data analysis method of claim 1, wherein each of the plurality of batches corresponds to a donor of cells comprised in the characteristic information.

13. A computer program stored on a non-transitory computer-readable storage medium, in combination with hardware, to execute the method of claim 1.

14. A device, comprising:
at least one processor configured to:
acquire characteristic information quantitatively measured from a single cell, corresponding to each of a plurality of batches;
calculate a conditional expectation and a conditional variance of the characteristic information modeled to a Poisson distribution, based on a sum of the characteristic information of cells corresponding to each of the plurality of batches;
calculate a covariance matrix of a residual matrix corresponding to the characteristic information, based on the conditional expectation, the conditional variance, and the characteristic information; and
calculate a PC value for a principal component analysis (PCA) of the residual matrix through eigen-decomposition of the covariance matrix.

15. The device of claim 14, wherein, for calculating the conditional expectation and the conditional variance, the processor is configured to:
model the characteristic information to a multinomial distribution conditional on the sum of the characteristic information of the cells corresponding to each of the plurality of batches; and
calculate the conditional expectation and the conditional variance based on the multinomial distribution of the characteristic information.

16. The device of claim 14, wherein, for calculating the covariance matrix of the residual matrix, the processor is configured to:
calculate a sum of AA^{T}, 2AB^{T}, and BB^{T},
wherein A denotes Y_{bij}/σ_{bij}; B denotes μ_{bij}/σ_{bij}; Y_{bij} denotes characteristic information corresponding to batch b, cell i, and gene j; μ_{bij} denotes a conditional expectation of Y_{bij}; and σ_{bij} denotes a conditional variance of Y_{bij}.

17. The device of claim 16, wherein the residual matrix is equivalent to a matrix obtained by subtracting B from A.

18. The device of claim 16, wherein AB^{T} and BB^{T} are calculated as expressed in the following equation,
$${n}_{bi} = {\sum }_{j} {Y}_{bij} , {n}_{bj} = {\sum }_{i ∈ {Φ}_{b}} {Y}_{bij} , {n}_{b} = {\sum }_{i ∈ {Φ}_{b , j}} {Y}_{bij} \\ {p}_{bi} = {n}_{bi} / {n}_{b} . \\ \begin{matrix}{AB}_{j {j}^{′}}^{T} = {\sum }_{b = 1}^{{n}_{B}} {\sum }_{k ∈ {Φ}_{b}} {A}_{bkj} {B}_{{bkj}^{′}} \\ = {\sum }_{b = 1}^{{n}_{B}} \sqrt{{n}_{{bj}^{′}}} {\sum }_{k ∈ {Φ}_{b}} {A}_{bkj} \sqrt{\frac{{p}_{bk}}{1 - {p}_{bk}}} \\ {BB}_{j {j}^{′}}^{T} = {\sum }_{b = 1}^{{n}_{B}} \sqrt{{n}_{bj} {n}_{{bj}^{′}}} {\sum }_{k ∈ {Φ}_{b}} \frac{{p}_{bk}}{1 - {p}_{bk}}\end{matrix} \qquad [ Equation ]$$
$${n}_{bi} = {\sum }_{j} {Y}_{bij} , {n}_{bj} = {\sum }_{i ∈ {Φ}_{b}} {Y}_{bij} , {n}_{b} = {\sum }_{i ∈ {Φ}_{b , j}} {Y}_{bij} \\ {p}_{bi} = {n}_{bi} / {n}_{b} . \\ \begin{matrix}{AB}_{j {j}^{′}}^{T} = {\sum }_{b = 1}^{{n}_{B}} {\sum }_{k ∈ {Φ}_{b}} {A}_{bkj} {B}_{{bkj}^{′}} \\ = {\sum }_{b = 1}^{{n}_{B}} {\sum }_{k ∈ {Φ}_{b}} {A}_{bkj} \sqrt{{n}_{{bj}^{′}} \frac{{p}_{bk}}{1 - {p}_{bk}}} \\ = {\sum }_{b = 1}^{{n}_{B}} \sqrt{{n}_{{bj}^{′}}} {\sum }_{k ∈ {Φ}_{b}} {A}_{bkj} \sqrt{\frac{{p}_{bk}}{1 - {p}_{bk}}} \\ {BB}_{j {j}^{′}}^{T} = {\sum }_{b = 1}^{{n}_{B}} \sqrt{{n}_{bj} {n}_{{bj}^{′}}} {\sum }_{k ∈ {Φ}_{b}} \frac{{p}_{bk}}{1 - {p}_{bk}}\end{matrix}$$

19. The device of claim 14, wherein, for calculating the covariance matrix of the residual matrix, the processor is configured to:
calculate a variance of a residual corresponding to each gene in the characteristic information, based on the conditional expectation, the conditional variance, and the characteristic information;
determine a subset of the characteristic information comprising at least a portion of genes comprised in the characteristic information, based on the variance of the residual corresponding to each gene; and
calculate a covariance matrix of a residual matrix corresponding to the determined subset.

20. The device of claim 19, wherein, for determining the subset of the characteristic information, the processor is configured to:
select a predetermined number of genes in an order of increasing variance of the residual; and
determine the subset of the characteristic information comprising the selected genes.
